(19) United States
(12) Patent Application Publication
(10) Pub. No.: US 2003/0181857 A1
(43) Pub. Date: Sep. 25, 2003

(54) Insufflation device with integral heater control

(76) Inventors: James Blake, Wolcottville, IN (US); Daniel L. Hayes, Kendallville, IN (US); Robert Einhaus, Fort Wayne, IN (US)

Correspondence Address: TAYLOR & AUST, P.C., 142 S. Main St., P.O. Box 560, Avilla, IN, 46710, US

(21) Appl. No.: 10/104,631

(22) Filed: Mar. 22, 2002

(51) Int. Cl. A61F 7/12

(52) U.S. Cl. 604/113; 604/26

(57) ABSTRACT

An insufflation device including a flexible tube, at least one resistive heating conductor having a first end and a second end, the at least one resistive heating conductor substantially disposed within the flexible tube and a temperature controller disposed within the flexible tube, the temperature controller electrically connected to the first end.

### BACKGROUND OF THE INVENTION

b 1. Field of the Invention

The present invention relates to an insufflation device, and, more particularly, to an insufflation device with an integral heater control.

2. Description of the Related Art

Surgical procedures that treat diseases and problems in the abdominal cavity have been plagued by high rates of morbidity and mortality throughout history. Physicians had often confined their intervention to indirect methods such as diet changes and purgatives. When confronted with a disease or abnormality requiring a more invasive approach, surgeons developed minimally invasive techniques that not only treated the disease or abnormality but also minimized patient morbidity.

In the modern era, surgeons have developed minimally invasive techniques including laproscopic insufflation techniques. The advantage of a minimally invasive approach as applied, for example, to gastrointestinal operations is that patients suffer less post operative pain, develop fewer infections and resume oral intake sooner than those surgical procedures performed through standard incisions.

Laproscopic insufflators are used to provide internal pressure to a body cavity during operations. A laproscropic insufflator may include a delivery gas path to deliver pressurized gas, normally carbon dioxide, to a patient's abdominal cavity. Laproscopic insufflator controls monitor the insufflation gas flow rates and internal pressures of gas delivery paths to the body cavity.

The flow of an insufflation gas, normally carbon dioxide, as well as the temperature and humidity of the insufflation gas are a concern of the surgeon for the well being of the patient. Excessive pressures can over inflate the cavity and dry insufflation gas can cause abnormal drying of the tissues. Insufflation gas, which is not appropriately prepared for the body cavity, can slow the healing process. Control systems, which monitor the gas pressure, gas, flow rate, humidity and temperature using remote sensors are utilized by surgeons and their staff to control these insufflation gas parameters.

What is needed in the art is a temperature control device, which does not require remote sensing.

### SUMMARY OF THE INVENTION

The present invention provides a heater control integral with an insufflation device.

The invention comprises, in one form thereof, an insufflation device including a flexible tube, at least one resistive heating conductor having a first end and a second end, the at least one resistive heating conductor substantially disposed within the flexible tube and a temperature controller disposed within the flexible tube, the temperature controller electrically connected to the first end.

An advantage of the present invention is that the heater control is integral with the insufflation device.

Another advantage is that no exterior control of the temperature of insufflation gas is necessary.

Yet another advantage is that the temperature control is located near the delivery point of insufflation gas.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a schematacized, partially sectioned side view of an embodiment of an insufflation device with an integral heater control of the present invention; and

FIG. 2 is a schematacized, partially sectioned side view of another embodiment of an insufflation device with an integral heater control of the present invention.

Corresponding reference characters indicate corresponding parts throughout the several views. The exemplifications set out herein illustrate one preferred embodiment of the invention, in one form, and such exemplifications are not to be construed as limiting the scope of the invention in any manner.

### DETAILED DESCRIPTION OF THE INVENTION

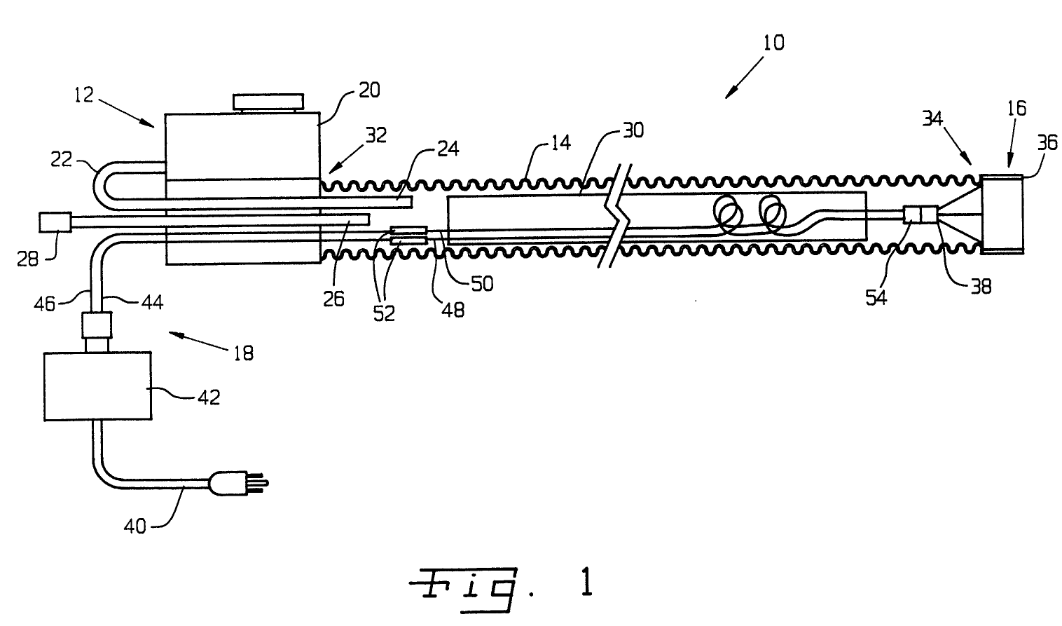

Referring now to the drawings, and more particularly to FIG. 1, there is shown an embodiment of the present invention including insufflation device 10 having input interface 12, flexible tubing 14, output interface 16 and heater system 18. Insufflation device 10 may be interconnected with other medical delivery systems to the patient and sources for electricity insufflation gas and water.

Input interface 12 includes water chamber 20, water tubing 22, wetting tube 24, distribution port 26 and gaseous input tube 28. Water chamber 20 is a reservoir for holding water to be delivered to insufflation gas within flexible tubing 14. Water chamber 20 may be made of a plastic with a filling port and a filler cap on the top thereof. Water tubing 22 connects water chamber 20 with wetting tube 24. Wetting tube 24 provides a pathway for water from water chamber 20 to an interior portion of flexible tubing 14. Distribution port 26 is the output for gas, normally carbon dioxide, and allows distribution of carbon dioxide within flexible tubing 14. Gaseous input tube 28 allows an interconnection with a gaseous source and delivers the insufflation gas to distribution port 26.

Flexible tubing 14 contains wick 30 therein and flexible tubing 14 has an ingress end 32 and an egress end 34. Flexible tubing 14 may be translucent or transparent to allow a visual check on moisture buildup within flexible tubing 14. Wick 30 may be of a woven fabric, which obtains moisture by way of wetting tube 24 and distributes moisture throughout the length of wick 30 to provide for the humidification of the insufflation gas. Ingress end 32 of flexible tubing 14 is connected to input interface 12. Egress end 34 of flexible tubing 14 is the output end for insufflation gas and is also a connected to output interface 16.

Output interface 16 includes delivery connection 36 and sensor positioning device 38. Delivery connection 36 is an interface allowing insufflation gas to pass therethrough to further surgical devices such as a verass needle. Sensor positioning device 38 may include plastic legs, which serve to hold a sensor central to a cross-section of flexible tube 14, thereby positioning sensor positioning device 38 in the midstream of the flow of insufflation gas.

Heating system 18 includes electrical cord 40, power conditioner 42, first conductor 44, second conductor 46, first resistive heater conductor 48, second resistive heater conductor 50, wire connections 52 and temperature controller 54. Electrical cord 40 provides electrical power to power conditioner 42. Power conditioner 42 includes a transformer for electrical isolation of insufflation device 10 from the power source. Power conditioner 42 is electrically connected to first conductor 44 and second conductor 46. First conductor 44 and second conductor 46 convey electrical power through input interface 12 into an interior portion of flexible tubing 14. First conductor 44 and second conductor 46 are respectively connected to first resistive heater conductor 48 and second resistive heater conductor 50 by way of wire connections 52. Changing from conductors 44 and 46 to resistive conductors 48 and 50 inside of flexible tubing 14 allow electrical energy to pass through input interface 12 without the generation of heat therein. First resistive heater conductor 48 and second resistive heater conductor 50 traverse the inside of flexible tubing 14 passing through the interior of wick 30 and electrically connecting to temperature controller 54. First resistive heater conductor 48 and second resistive heater conductor 50 may traverse flexible tubing 14 in a circuitous manner in order to distribute heat within flexible tubing 14.

Temperature controller 54 is positioned upon sensor positioning device 38 approximately equal distance from the inner surface of flexible tubing 14. Temperature controller 54 is proximate egress end 34 of flexible tubing 14. Temperature controller 54 may be a positive temperature coefficient (PTC) device 54, which reacts to the surrounding temperature of the insufflation gas and controls the current flow through resistive heater conductors 48 and 50. The control of current through resistive heater conductors 48 and 50 provide a controlled temperature at egress end 34 of insufflation device 10.

Temperature controller 54 in the form of PTC device 54 detects the temperature and the resistance is modified based on the temperature of the PTC device 54. PTC device 54 has a small mass thereby allowing a quick reaction to the surrounding temperature of the insufflation gas passing through flexible tubing 14.

Alternatively, more than one circuit element may supply heat to the insufflation gas. And, more than one PTC device 54 may be utilized to control those circuit elements.

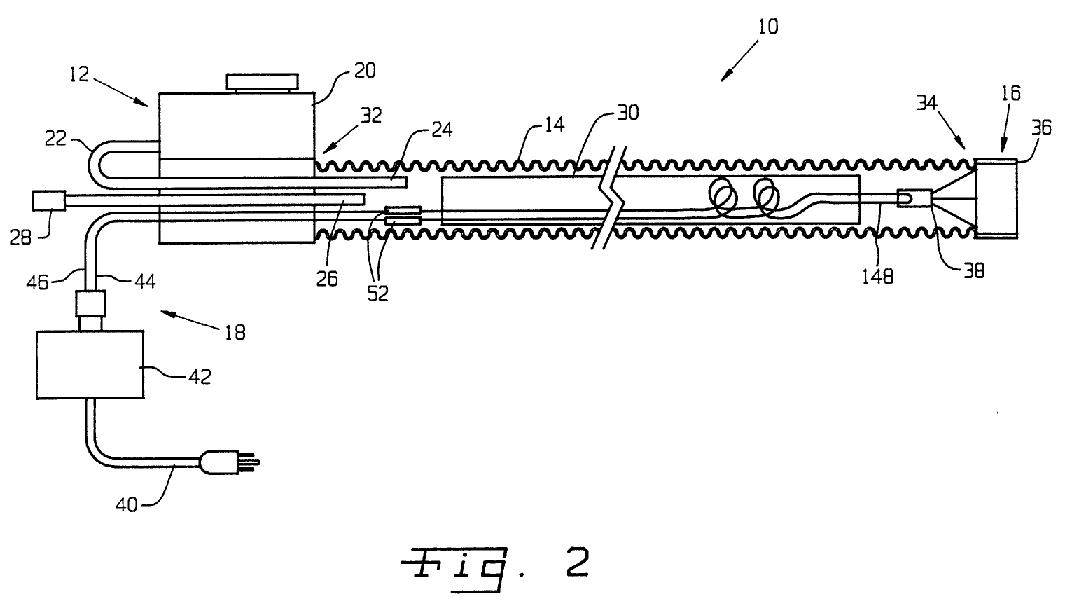

Now, additionally referring to FIG. 2, there is shown another embodiment of the present invention. The illustration of this embodiment is substantially similar to the previous embodiment and corresponding numbers are used therein.

Heating system 18 includes electrical cord 40, power conditioner 42, first conductor 44, second conductor 46, resistive heater conductor 148 and wire connections 52. Electrical cord 40 provides electrical power to power conditioner 42. Power conditioner 42 includes a transformer for electrical isolation of insufflation device 10 from the power source. Power conditioner 42 is electrically connected to first conductor 44 and second conductor 46. First conductor 44 and second conductor 46 convey electrical power through input interface 12 into an interior portion of flexible tubing 14. First conductor 44 and second conductor 46 are connected to respective ends of resistive heater conductor 148 by way of wire connections 52. Resistive heater conductor 148 traverses the inside of flexible tubing 14 passing through the interior of wick 30. Resistive heater conductor 148 may traverse flexible tubing 14 in a circuitous manner in order to distribute heat within flexible tubing 14.

Selecting the composition of the conductive material, the cross-sectional area and the length of resistive heater conductor 148 controls the power dissipation therein. For example, resistive heater conductor 148 may be made of a positive temperature coefficient material, which inherently regulates the heat generated by resistive heater conductor 148. As the temperature of resistive heater conductor 148 rises its resistance rises, which restricts the current therethrough thereby reducing the power dissipated to the insufflation gas. As the insufflation gas flow and temperature varies, the conduction of heat thereto proportionally varies since resistive heater conductor 148 increasingly dissipates more heat when more heat is transferred to the insufflation gas and less heat when less heat is transferred to the insufflation gas.

Alternatively, resistive heating conductor 148 of this embodiment or temperature controller 54 of the previous embodiment may include a thermal disconnection property that will disconnect the flow of electrical current through resistive heating conductor 148, or 48 and 50, respectively. The property may be in the form of a permanent disconnection, such as a fusible link or as a resetable link. A resetable link may include the ability for it to automatically reset or to be manually resetable.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An insufflation heater, comprising:
a flexible tube;
at least one resistive heating conductor positioned within said tube and having a first end and a second end; and
a temperature controller positioned within said tube and connected to said first end.

2. The heater of claim 1, wherein said temperature controller is a positive temperature coefficient device.

3. The heater of claim 1, further comprising a plurality of electrical conductors, one of said plurality of electrical conductors electrically connected to said second end.

4. The heater of claim 3, wherein said at least one resistive heating conductor includes a first resistive heating conductor and a second resistive heating conductor, said first ends of said first and said second resistive heating conductors being electrically connected to said temperature controller, said plurality of electrical conductors including a first electrical conductor and a second electrical conductor, said first electrical conductor electrically connected to said second end of said first resistive heating conductor, said second electrical conductor electrically connected to said second end of said second resistive heating conductor.

5. The heater of claim 4, wherein said tube includes an egress end and said temperature controller is a positive temperature coefficient device positioned in association with said egress end.

6. An insufflation device, comprising:
a flexible tube;
at least one resistive heating conductor having a first end and a second end, said at least one resistive heating conductor substantially disposed within said flexible tube; and
a temperature controller disposed within said flexible tube, said temperature controller electrically connected to said first end.

7. The device of claim 6, wherein said temperature controller is a positive temperature coefficient device.

8. The device of claim 6, further comprising a plurality of electrical conductors, one of said plurality of electrical conductors electrically connected to said second end.

9. The device of claim 8, wherein said at least one resistive heating conductor includes a first resistive heating conductor and a second resistive heating conductor, said first ends of said first and said second resistive heating conductors being electrically connected to said temperature controller, said plurality of electrical conductors including a first electrical conductor and a second electrical conductor, said first electrical conductor electrically connected to said second end of said first resistive heating conductor, said second electrical conductor electrically connected to said second end of said second resistive heating conductor.

10. The device of claim 9, wherein said temperature controller is a positive temperature coefficient device.

11. The device of claim 6, wherein said flexible tube has an ingress end and an egress end, said temperature controller being disposed proximate said egress end.

12. The device of claim 11, wherein said temperature controller is centrally disposed within said flexible tube.

13. The device of claim 11, further comprising a gas and water input interface connected proximate said ingress end.

14. The device of claim 6, further comprising a wick disposed within said flexible tube, at least one of said at least one resistive heating conductors being at least partially disposed within said wick.

15. An insufflation device, comprising:
a flexible tube having an ingress end and an egress end;
a plurality of resistive heating conductors including a first resistive heating conductor and a second resistive conductor, both first and second resistive heating conductors having a first end and a second end, both said first and said second resistive heating conductors substantially disposed within said flexible tube; and
a positive temperature coefficient controller disposed within said flexible tube, said positive temperature coefficient controller electrically connected to said first ends of both said first and said second resistive conductors, said positive temperature coefficient controller being disposed proximate said egress end.

16. An insufflation device, comprising:
a flexible tube; and
at least one resistive heating conductor assembly at least substantially disposed within said flexible tube, said at least one resistive heating conductor assembly including a temperature controller.

17. The device of claim 16, wherein said at least one resistive heating conductor assembly includes at least one resistive heating conductor, said temperature controller comprising a property of at least one said resistive heating conductor which one of permanently disconnects and temporarily disconnects electrical power therethrough.
